(19) United States
(12) Patent Application Publication
(10) Pub. No.: US 2025/0134391 A1
(43) Pub. Date: May 1, 2025

(54) HUMAN BODY SURFACE TEMPERATURE CALCULATION SYSTEM, HUMAN BODY SURFACE TEMPERATURE CALCULATION METHOD, AND RECORDING MEDIUM

(71) Applicant: Panasonic Intellectual Property Management Co., Ltd., Osaka (JP)

(72) Inventors: Kazuo ITOH, Osaka (JP); Tatsuo KOGA, Osaka (JP)

(21) Appl. No.: 18/834,913

(22) PCT Filed: Feb. 2, 2023
(86) PCT No.: PCT/JP2023/003310

§ 371 Date: Jul. 31, 2024

(30) Foreign Application Priority Data
Mar. 28, 2022 (JP) 2022-052127

(51) Int. Cl. A61B 5/01; A61B 5/00

(52) U.S. Cl. CPC A61B 5/015; A61B 5/0075; A61B 5/7267; A61B 5/7278; A61B 2576/00

(57) ABSTRACT

A human body surface temperature calculation system includes: an infrared sensor; an obtainer that obtains temperature distribution data indicating a temperature distribution in a target space which is obtained by the infrared sensor; a thermal image generator that generates a thermal image of the target space based on the temperature distribution data obtained by the obtainer; an extractor that extracts, using a machine learning model, a human region indicating a person captured in the thermal image; and a calculator that extracts a temperature value group corresponding to the human region from the temperature distribution data and calculates a body surface temperature of the person based on the temperature value group extracted.

### CROSS-REFERENCE OF RELATED APPLICATIONS

This application is the U.S. National Phase under 35 U.S.C. § 371 of International Patent Application No. PCT/JP2023/003310, filed on Feb. 2, 2023, which in turn claims the benefit of Japanese Patent Application No. 2022-052127, filed on Mar. 28, 2022, the entire disclosure of which Applications are incorporated by reference herein.

### TECHNICAL FIELD

The present invention relates to a human body surface temperature calculation system, a human body surface temperature calculation method, and a recording medium.

### BACKGROUND ART

There are known methods of estimating the clothing amount of a clothing portion of a subject captured in a thermal image that indicates a temperature distribution in a target space, based on the temperature of the clothing portion and clothing information about clothing, to estimate the sensible temperature of a user (for example, Patent Literature (PTL) 1).

### CITATION LIST

### Patent Literature

[PTL 1] International Publication No. 2020/084777

### SUMMARY OF INVENTION

### Technical Problem

The present invention provides a human body surface temperature calculation system, a human body surface temperature calculation method, and a recording medium that can accurately calculate the body surface temperature of a person present in a target room.

### Solution to Problem

A human body surface temperature calculation system according to one aspect of the present invention includes: an infrared sensor; an obtainer that obtains temperature distribution data indicating a temperature distribution in a target space, the temperature distribution data being obtained by the infrared sensor; a thermal image generator that generates a thermal image of the target space based on the temperature distribution data obtained by the obtainer; an extractor that extracts, using a machine learning model, a human region indicating a person captured in the thermal image; and a calculator that extracts a temperature value group corresponding to the human region from the temperature distribution data and calculates a body surface temperature of the person based on the temperature value group extracted.

A human body surface temperature calculation method according to one aspect of the present invention includes: obtaining temperature distribution data indicating a temperature distribution in a target space; generating a thermal image of the target space based on the temperature distribution data obtained in the obtaining; extracting, using a machine learning model, a human region indicating a person captured in the thermal image; and extracting a temperature value group corresponding to the human region from the temperature distribution data and calculating a body surface temperature of the person based on the temperature value group extracted.

A recording medium according to one aspect of the present invention is a non-transitory computer-readable recording medium for use in a computer, the recording medium having recorded thereon a computer program for causing the computer to execute the above-described human body surface temperature calculation method.

### Advantageous Effects of Invention

A human body surface temperature calculation system, a human body surface temperature calculation method, and a recording medium according to the present invention can accurately calculate the body surface temperature of a person present in a target space.

### BRIEF DESCRIPTION OF DRAWINGS

FIG. 1 is a block diagram illustrating a functional configuration of a human body surface temperature calculation system according to an embodiment.

FIG. 2 is a flowchart illustrating an example of operations performed by the human body surface temperature calculation system according to the embodiment.

FIG. 3 is a schematic diagram illustrating the flow shown in FIG. 2.

FIG. 4 is a diagram illustrating one example of a thermal image.

FIG. 5 is a flowchart illustrating Example 1 of the detailed flow of step S05 shown in FIG. 2.

FIG. 6 is a flowchart illustrating Example 2 of the detailed flow of step S05 shown in FIG. 2.

FIG. 7 is a diagram illustrating another example of the thermal image.

FIG. 8 is a flowchart illustrating Example 3 of the detailed flow of step S05 shown in FIG. 2.

FIG. 9 is a flowchart illustrating Example 4 of the detailed flow of step S05 shown in FIG. 2.

### DESCRIPTION OF EMBODIMENT

Hereinafter, embodiments will be described with reference to the drawings. Note that the embodiments below each describe a general or specific example. The numerical values, shapes, materials, elements, the arrangement and connection of the elements, steps, orders of the steps, etc. presented in the embodiments below are mere examples, and are not intended to limit the present invention. Furthermore, among the elements in the embodiments below, those not recited in any one of the independent claims will be described as optional elements.

Note that the drawings are schematic diagrams, and do not necessarily provide strictly accurate illustration. Throughout the drawings, the same reference sign is given to substantially the same element, and redundant description may be omitted or simplified.

### EMBODIMENT

### Configuration

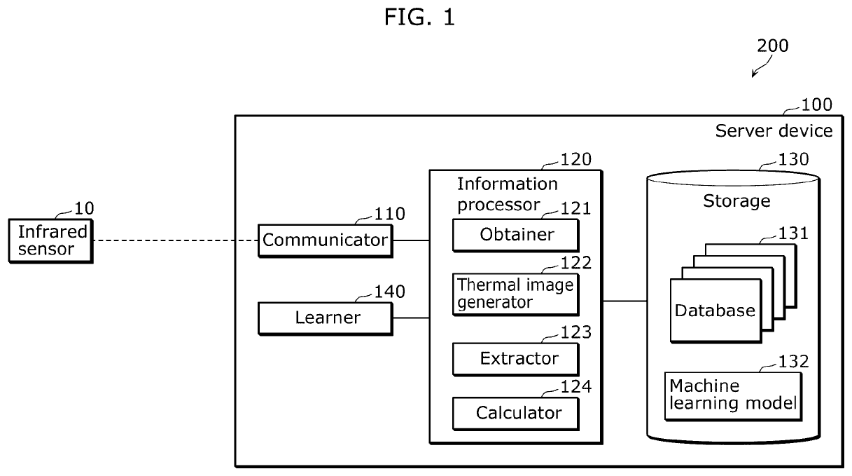

First, the configuration of a human body surface temperature calculation system according to an embodiment will be described. FIG. 1 is a block diagram illustrating a functional configuration of the human body surface temperature calculation system according to the embodiment.

Human body surface temperature calculation system 200 generates a thermal image based on temperature distribution data of a target space obtained from infrared sensor 10, extracts, using a machine learning model, a human region indicating a person captured in the thermal image, and calculates the body surface temperature of the person based on a temperature value group corresponding to the human region. The target space is, for example, an office space, but may be an indoor space in other facilities such as a space in a commercial facility and a space in a home. As illustrated in FIG. 1, human body surface temperature calculation system 200 includes infrared sensor 10 and server device 100. Note that human body surface temperature calculation system 200 may include several infrared sensors 10.

### Infrared Sensor

Infrared sensor 10 is, for example, provided on the ceiling or the like of the target space to obtain temperature distribution data indicating a temperature distribution in the target space when the target space is viewed from above. Note that infrared sensor 10 may generate a thermal image based on the obtained temperature distribution data. Infrared sensor 10 may be an infrared array sensor (thermal image sensor) including an array of M×L infrared detection elements. Stated differently, the temperature distribution data to be obtained by infrared sensor 10 is a matrix of temperature values arranged in M columns×L rows, where M and N each are an integer of two or more. Accordingly, a thermal image to be generated based on the temperature distribution data includes M×L pixels. The thermal image shows the temperature distribution in a sensing range sensed by infrared sensor 10, in the resolution of M×L.

For example, infrared sensor 10 may be removably coupled to a power supply terminal included in a lighting device provided on the ceiling of the target space. In this case, infrared sensor 10 receives the supply of power from the lighting device to operate. The power supply terminal is, for example, a universal serial bus (USB) terminal. Moreover, infrared sensor 10 may be directly secured to the ceiling of the target space not via the lighting device. Infrared sensor 10 may also be secured to a wall or the like to generate a thermal image showing a temperature distribution of the target space when the target space is viewed from a side.

### Server Device

Server device 100 generates a thermal image based on temperature distribution data obtained from infrared sensor 10, extracts, using a machine learning model, a human region indicating a person captured in the thermal image, extracts a temperature value group corresponding to the human region from the temperature distribution data, and calculates the body surface temperature of the person based on the temperature value group. Server device 100 is an edge computer provided in a facility (building) that includes a target space, but server device 100 may be a cloud computer provided outside the facility. Server device 100 includes, for example, communicator 110, information processor 120, storage 130, and learner 140.

Communicator 110 is a communication module (communication circuit) for server device 100 to communicate with infrared sensor 10. Communicator 110 receives temperature distribution data of a target space from infrared sensor 10, for example. The communication performed by communicator 110 may be wireless communication or wired communication. The communication standard used for the communication is not particularly limited.

Information processor 120 obtains temperature distribution data received by communicator 110, generates a thermal image based on the obtained temperature distribution data, extracts a human region from the generated thermal image, and performs information processing for calculating the body surface temperature of a person based on a temperature value group corresponding to the extracted human region. More specifically, information processor 120 is implemented by a processor or a microcomputer. More specifically, information processor 120 includes obtainer 121 that obtains temperature distribution data of a target space which is received by communicator 110, thermal image generator 122 that generates a thermal image of a target region based on the obtained temperature distribution data, extractor 123 that extracts, using machine learning model 132, a human region indicating a person captured in the thermal image, and calculator 124 that extracts a temperature value group corresponding to the human region from the temperature distribution data and calculates the body surface temperature of the person based on the extracted temperature value group. The functions of obtainer 121, thermal image generator 122, extractor 123, and calculator 124 are implemented by the processor or microcomputer included in information processor 120 executing computer programs stored in storage 130. The detailed function of each of obtainer 121, thermal image generator 122, extractor 123, and calculator 124 will be described later.

Storage 130 is a storage device that stores temperature distribution data received by communicator 110, computer programs and the like to be executed by information processor 120. Storage 130 also stores database 131, machine learning model 132, etc. Storage 130 may store training data (not illustrated) to be used for training machine learning model 132. More specifically, storage 130 is implemented by a semiconductor memory, a hard disk drive (HDD), or the like.

Database 131 contains, for example, temperature distribution data, a temperature value group corresponding to a human region, a high-temperature value group corresponding to a high-temperature object region, a low-temperature value group corresponding to a low-temperature object region, etc.

Machine learning model 132 uses a thermal image as an input, and outputs a human region indicating a person captured in the thermal image. The human region is a region surrounded by the outline of the person captured in the thermal image. Machine learning model 132 is to be a machine learning model including a convolution layer, and may be, for example, a convolutional neural network (CNN). However, machine learning model 132 is not limited to the foregoing. Moreover, machine learning model 132 may include two or more models. For example, machine learning model 132 may include a first machine learning model that uses a thermal image as an input to output a super-resolution image, and a second machine learning model that uses the super-resolution image output from the first machine learning model as an input to output a human region. The first machine learning model may be, for example, a generative adversarial network for super-resolution (SRGAN) or a super-resolution convolutional neural network (SRCNN). The second machine learning model may be, for example, a region-convolutional neural network (R-CNN), you only look at once (YOLO), or a single shot multibox detector (SSD). Note that the above-mentioned models are mere examples, and thus the first machine learning model and second machine learning model are not limited to these models. In addition, as described above, different types of machine learning models may be adopted for the first machine learning model and second machine learning model, but the same type of machine learning models may be adopted for the first machine learning model and second machine learning model.

The training data includes a thermal image showing a temperature distribution in a target space as input data, and a human region indicating a person captured in the thermal image as output data. More specifically, the training data may be a data set including a set of a thermal image as input data and a human region as output data, or may include several data sets (e.g., first training data and second training data). For example, the first training data is used for training the first machine learning model, and is, for example, a dataset including a set of a thermal image as input data and a super-resolution thermal image as output data. The super-resolution thermal image is obtained by increasing the resolution of the thermal image. In addition, the second training data is, for example, used for training the second machine learning model, and is a data set including a set of the super-resolution thermal image as input data and a human region as output data. Note that machine learning model 132 may be an artificial intelligence (AI) model.

Learner 140 uses training data to perform machine learning. Through machine learning, learner 140 generates a machine learning model that uses a thermal image showing a temperature distribution in a target space as an input and outputs a human region indicating a person captured in the thermal image. Learner 140 stores a trained machine learning model into storage 130 to update machine learning model 132. Learner 140 is implemented by, for example, a processor executing a program stored in storage 130.

Note that although FIG. 1 illustrates an example of updating machine learning model 132 by learner 140 of server device 100 generating a trained machine learning model and storing the generated trained machine learning model into storage 130, updating machine learning model 132 is not limited to the foregoing example. For example, a trained machine learning model may be generated in a cloud server provided outside a building, and the cloud server may transmit the trained machine learning model to server device 100 to update machine learning model 132.

### Operation Example

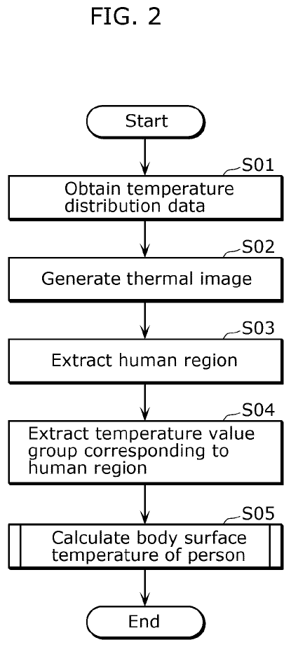

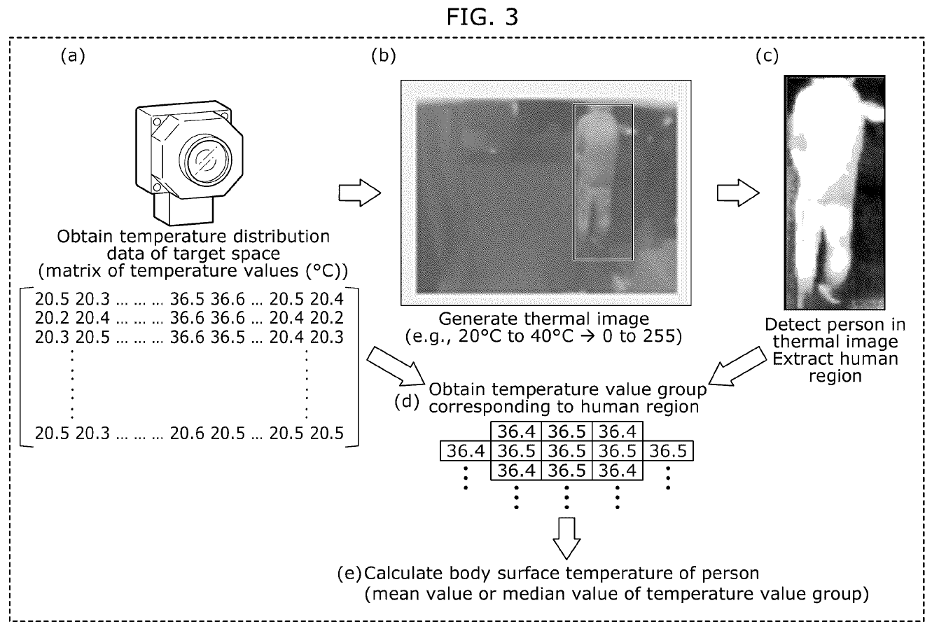

Next, operations performed by human body surface temperature calculation system 200 will be described. FIG. 2 is a flowchart illustrating an example of operations performed by human body surface temperature calculation system 200. FIG. 3 is a schematic diagram illustrating the flow shown in FIG. 2.

Communicator 110 of server device 100 receives temperature distribution data from infrared sensor 10 (not illustrated). At this time, information processor 120 stores the received temperature distribution data into storage 130 (not illustrated).

Next, obtainer 121 obtains the temperature distribution data received by communicator 110 and stored into storage 130 (S01), and outputs the temperature distribution data to thermal image generator 122. Thermal image generator 122 generates a thermal image of a target space, based on the obtained temperature distribution data (S02). For example, as illustrated in (a) of FIG. 3, the temperature distribution data obtained in step S01 is a matrix of temperature values (also called the temperature quantities). In addition, as illustrated in (b) of FIG. 3, the thermal image generated in step S02 is an 8-bit image obtained by converting, for example, the temperature range of from 20° C. to 40° C. into 0 to 255 tones.

Next, extractor 123 extracts, using machine learning model 132, a human region indicating a person in the thermal image (S03). To be more specific, as illustrated in (b) and (c) of FIG. 3, extractor 123 segments, using machine learning model 132, the thermal image and performs a classification process to classify a region indicating a person captured in the segmented thermal image as a class of human. In this way, extractor 123 can detect a person in the thermal image to extract a region indicating the detected person (a region surrounded by the outline of the person). The segmentation may be semantic segmentation or instance segmentation. Note that extractor 123 may convert the thermal image into a high-resolution image or need not convert the thermal image into a high-resolution image. For example, extractor 123 may convert, using the first machine learning model, the thermal image into a super-resolution image, or may convert the thermal image into a high-resolution image by calculating the mean value of pixel values of adjacent pixels to insert, between these adjacent pixels, a new pixel having a pixel value corresponding to the mean value. Extractor 123 performs, using machine learning model 132, a human detection process on the thermal image to detect a person in the thermal image, and extracts a region surrounded by the outline of the detected person (the so-called human region).

Next, calculator 124 extracts a temperature value group corresponding to the human region from the temperature distribution data (S04) and calculates the body surface temperature of the person based on the extracted temperature value group (S05). To be more specific, as illustrated in (d) and (e) of FIG. 3, calculator 124 may extract, from the matrix of the temperature distribution data, temperature values corresponding to pixels within the human region in the thermal image, and may calculate the mean value or median value of the extracted temperature value group to calculate the body surface temperature of the person.

Next, calculator 124 outputs a calculation result (not illustrated). More specifically, calculator 124 outputs, as the calculation result, the body surface temperature of the person present in the target space. Calculator 124 may output, as the calculation result, information indicating coordinates of the person in addition to the body surface temperature of the person. Note that the calculation results are stored in storage 130.

As has been described above, human body surface temperature calculation system 200 can (i) detect a person present in a target space, (ii) calculate the body surface temperature of the detected person, and (iii) output a calculation result.

The output calculation result may be provided to, for example, a control device (not illustrated) that controls a device such as an air conditioner. In this way, the control device can control a device based on the body surface temperature of a person present in a target space.

### Specific Example of Calculation Step

In the above-described operation example, the calculation step (S05 in FIG. 2) was exemplified as detecting a person captured in a thermal image to extract a human region indicating the detected person, and calculating the body surface temperature of the person based on a temperature value group corresponding to the extracted human region. Hereinafter, a human body surface temperature calculation method that is employed when a person and a heat source overlapping each other are captured in a thermal image will be described in detail.

### Example 1

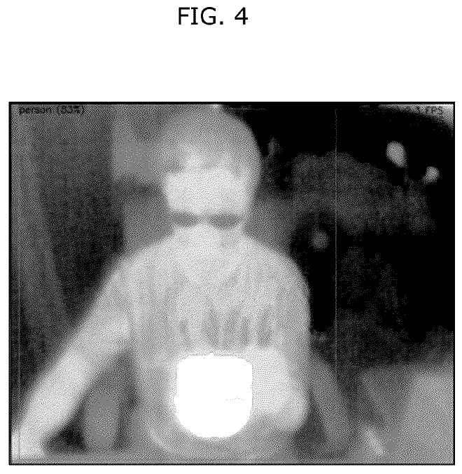

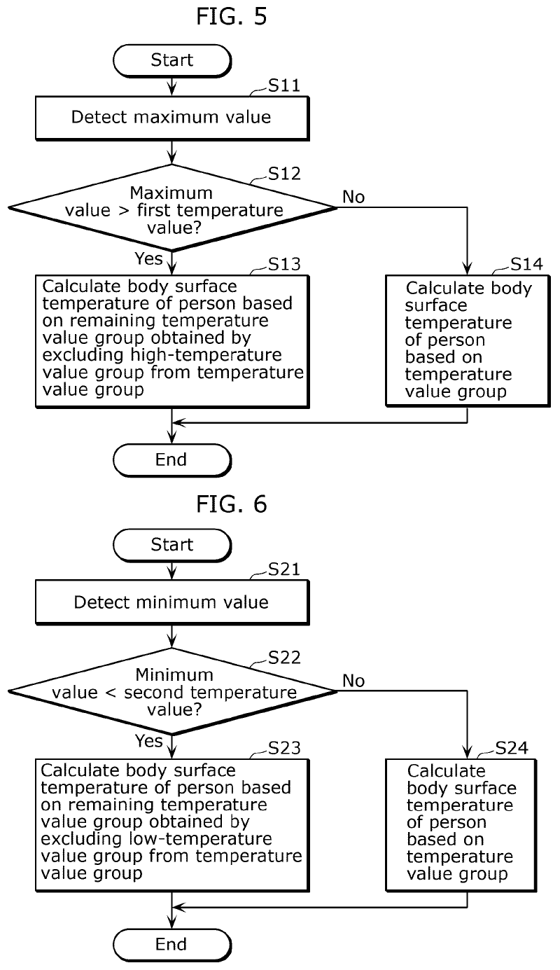

In Example 1, an example of a process that is performed when a person and a single heat source overlapping each other are captured in a thermal image will be described. FIG. 4 is a diagram illustrating one example of the thermal image. FIG. 5 is a flowchart illustrating Example 1 of the detailed flow of step S05 shown in FIG. 2.

Calculator 124 detects the maximum value of a temperature value group corresponding to the human region extracted in step S04 in FIG. 2 (S11) to determine whether the detected maximum value exceeds a first temperature value (e.g., 50° C.) (S12). Note that the temperature value group corresponding to the human region is a group of temperature values corresponding to the pixel values of respective pixels indicating the human region in a thermal image.

When calculator 124 determines that the maximum value exceeds the first temperature value (Yes in S12), calculator 124 identifies (not illustrated) a high-temperature value group of temperature values greater than or equal to a difference between the maximum value and the first value (e.g., 10° C.). The high-temperature value group corresponds to a high-temperature object region indicating a high-temperature object different from the person. The high-temperature object is, for example, a hot beverage such as hot coffee, hot food, a heating pad, etc. Next, calculator 124 calculates the body surface temperature of the person based on the remaining temperature value group obtained by excluding the high-temperature value group from the temperature value group corresponding to the human region (S13). To be more specific, calculator 124 may calculate the mean value or median value of the remaining temperature value group obtained by excluding the high-temperature value group from the temperature value group corresponding to the human region to calculate the body surface temperature of the person.

Alternatively, when calculator 124 determines that the maximum value does not exceed the first temperature value (No in S12), calculator 124 calculates the body surface temperature of the person based on the temperature value group corresponding to the human region (S14). In this case, the body surface temperature of the person to be calculated may be the mean value or median value of the temperature value group.

As has been described above, when a high-temperature object region is present within a human region (i.e., when a person and a high-temperature object overlapping each other are captured in a thermal image), human body surface temperature calculation system 200 can exclude a high-temperature value group corresponding to the high-temperature object region from a temperature value group corresponding to the human region. In this way, human body surface temperature calculation system 200 is not easily affected by a heat source other than a person, such as a high-temperature object present within a human region, in calculating the body surface temperature of the person. Therefore, human body surface temperature calculation system 200 can very accurately calculate the body surface temperature of a person.

### Example 2

In Example 2, an example of a process that is performed when a person and a low-temperature heat source overlapping each other are captured in a thermal image will be described. FIG. 6 is a flowchart illustrating Example 2 of the detailed flow of step S05 shown in FIG. 2.

Calculator 124 detects the minimum value of the temperature value group corresponding to the human region extracted in step S04 in FIG. 2 (S21) to determine whether the detected minimum value falls below a second temperature value (e.g., 10° C.) (S22).

When calculator 124 determines that the minimum value falls below the second temperature value (Yes in S22), calculator 124 identifies (not illustrated) a low-temperature value group of temperature values less than or equal to a sum of the minimum value and a second value (e.g., 20° C.). The low-temperature value group corresponds to a low-temperature object region indicating a low-temperature object different from the person. The low-temperature object is, for example, a cold beverage such as iced coffee, cold food such as an ice cream, an ice pack, etc. Next, calculator 124 calculates the body surface temperature of the person based on the remaining temperature value group obtained by excluding the low-temperature value group from the temperature value group corresponding to the human region (S23). To be more specific, calculator 124 may calculate the mean value or median value of the remaining temperature value group obtained by excluding the low-temperature value group from the temperature value group corresponding to the human region to calculate the body surface temperature of the person.

Alternatively, when calculator 124 determines that the minimum value does not fall below the second temperature value (No in S22), calculator 124 calculates the body surface temperature of the person based on the temperature value group corresponding to the human region (S24). In this case, the body surface temperature of the person to be calculated may be the mean value or median value of the temperature value group.

As has been described above, when a low-temperature object region is present within a human region (i.e., when a person and a low-temperature object overlapping each other are captured in a thermal image), human body surface temperature calculation system 200 can exclude a low-temperature value group corresponding to the low-temperature object region from a temperature value group corresponding to the human region. In this way, human body surface temperature calculation system 200 is not easily affected by a heat source other than a person, such as a low-temperature object present within a human region, in calculating the body surface temperature of the person. Therefore, human body surface temperature calculation system 200 can very accurately calculate the body surface temperature of a person.

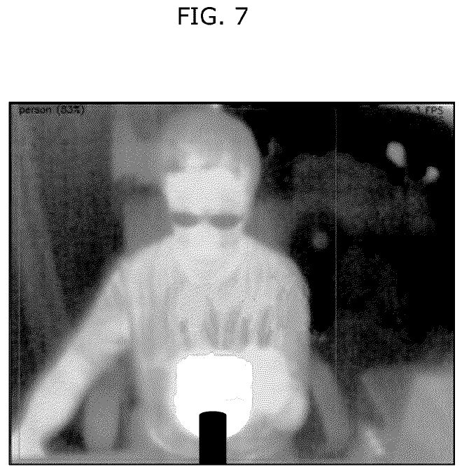

Note that Examples 1 and 2 of the calculation processes may be performed in parallel. FIG. 7 is a diagram illustrating another example of the thermal image. FIG. 7 shows an example in which a person, a high-temperature heat source, and a low-temperature heat source overlapping each other are captured in a thermal image.

For example, calculator 124 detects the maximum value and minimum value within a temperature value group corresponding to a human region, and determines whether the maximum value exceeds a first temperature value and the minimum value falls below a second temperature value.

Next, when the maximum value exceeds the first temperature value and the minimum value falls below the second temperature value, calculator 124 calculates the body surface temperature of a person based on the remaining temperature value group obtained by excluding a high-temperature value group and a low-temperature value group from the temperature value group corresponding to the human region.

Note that when the maximum value exceeds the first temperature value and the minimum value does not fall below the second temperature value, calculator 124 calculates the body surface temperature of the person based on the remaining temperature value group obtained by excluding the high-temperature value group from the temperature value group corresponding to the human region.

Note that when the maximum value does not exceed the first temperature value and the minimum value falls below the second temperature value, calculator 124 calculates the body surface temperature of the person based on the remaining temperature value group obtained by excluding the low-temperature value group from the temperature value group corresponding to the human region.

As has been described above, when a high-temperature object region and a low-temperature object region are present within a human region (i.e., when a person, a high-temperature object, and a low-temperature object overlapping each other are captured in a thermal image), human body surface temperature calculation system 200 can exclude a high-temperature value group and a low-temperature value group from a temperature value group corresponding to the human region. In this way, human body surface temperature calculation system 200 is not easily affected by a heat source other than a person, such as the high-temperature object or low-temperature object present within the human region, in calculating the body surface temperature of the person. Therefore, human body surface temperature calculation system 200 can very accurately calculate the body surface temperature of a person.

### Example 3

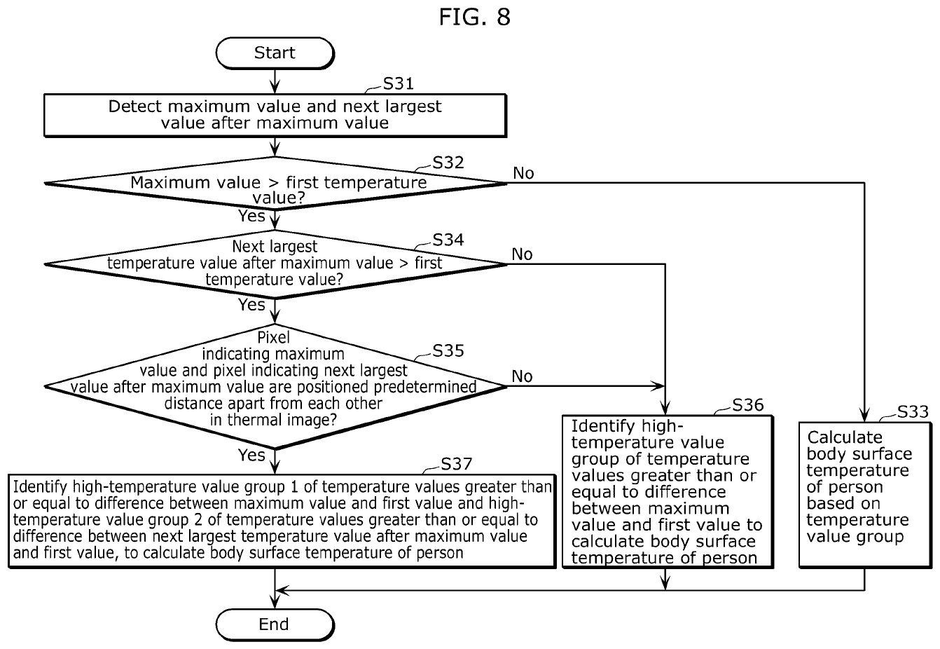

In Example 1, an example of a process that is performed when a person and a single heat source overlapping each other are captured in a thermal image was described. In Example 3, an example of a process that is performed when a person and several (e.g., two) high-temperature heat sources overlapping each other are captured in a thermal image will be described. FIG. 8 is a flowchart illustrating Example 3 of the detailed flow of step S05 shown in FIG. 2.

Calculator 124 detects the maximum value and the next largest temperature value after the maximum value of the temperature value group corresponding to the human region extracted in step S04 in FIG. 2 (S31). Calculator 124 determines whether the maximum value exceeds a first temperature value (e.g., 50° C.) (S32). When calculator 124 determines that the maximum value does not exceed the first temperature value (No in S32), calculator 124 calculates the body surface temperature of the person based on the temperature value group corresponding to the human region (S33). Alternatively, when calculator 124 determines that the maximum value exceeds the first temperature value (Yes in S32), calculator 124 determines whether the next largest temperature value after the maximum value exceeds the first temperature value (S34).

When calculator 124 determines that the next largest temperature value after the maximum value does not exceed the first temperature value (No in S34), calculator 124 identifies (not illustrated) a high-temperature value group of temperature values greater than or equal to a difference between the maximum value and a first value (e.g., 10° C.), and calculates the body surface temperature of the person based on the remaining temperature value group obtained by excluding the high-temperature value group from the temperature value group (S36). Alternatively, when calculator 124 determines that the next largest temperature value after the maximum value exceeds the first temperature value (Yes in S34), calculator 124 determines whether a pixel indicating the maximum value and a pixel indicating the next largest temperature value after the maximum value are positioned a predetermined distance apart from each other in a thermal image (S35). Note that the predetermined distance may be, for example, at least 10% of the number of pixels in the transverse direction of a thermal image and/or may be at least 10% of the number of pixels in the longitudinal direction of the thermal image. For example, the predetermined distance will be described in detail with reference to FIG. 7. Since the above-mentioned predetermined distance changes according to a distance between an infrared sensor and a heat source, the above-mentioned predetermined distance is to be a distance that exceeds the size (e.g., the width for the width direction and the height for the longitudinal direction) of each of a high-temperature heat source (e.g., a hot beverage) and a low-temperature heat source (e.g., a cold, bottled beverage) captured in a thermal image.

When calculator 124 determines that the pixel indicating the maximum value and the pixel indicating the next largest temperature value after the maximum value are not positioned the predetermined distance apart from each other (stated differently, not spaced apart with the predetermined space therebetween) in the thermal image (No in S35), calculator 124 identifies (not illustrated) a high-temperature value group of temperature values greater than or equal to a difference between the maximum value and the first value (e.g., 10° C.), and calculates the body surface temperature of the person based on the remaining temperature value group obtained by excluding the high-temperature value group from the temperature value group (S36). Alternatively, when calculator 124 determines that the pixel indicating the maximum value and the pixel indicating the next largest temperature value after the maximum value are positioned the predetermined distance apart from each other (stated differently, spaced apart with the predetermined space therebetween) in the thermal image (Yes in S35), calculator 124 identifies (not illustrated) high-temperature value group 1 of temperature values greater than or equal to a difference between the maximum value and the first value (e.g., 10° C.), further identifies (not illustrated) high-temperature value group 2 of temperature values greater than or equal to a difference between the next largest temperature value after the maximum value and the first value (e.g., 10° C.), and calculates the body surface temperature of the person based on the remaining temperature value group obtained by excluding high-temperature value group 1 and high-temperature value group 2 from the temperature value group (S37).

As has been described above, when several (here, two) high-temperature object regions are present within a human region (i.e., when several high-temperature objects overlapping each other are captured in a thermal image), human body surface temperature calculation system 200 can identify the high-temperature value group of each of the several high-temperature object regions to exclude these high-temperature value groups from a temperature value group corresponding to the human region. In this way, even though several heat sources different from a person are present in a thermal image, human body surface temperature calculation system 200 is not easily affected by these heat sources in calculating the body surface temperature of the person. Therefore, human body surface temperature calculation system 200 can accurately calculate the body surface temperature of a person.

### Example 4

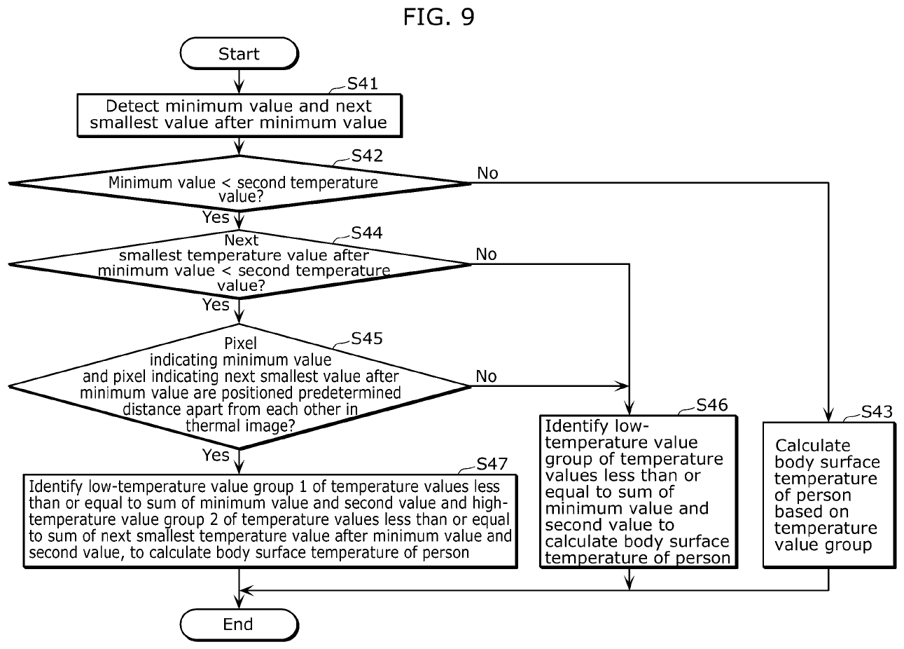

In Example 2, an example of a process that is performed when a person and a single low-temperature heat source overlapping each other are captured in a thermal image was described. In Example 4, an example of a process that is performed when a person and several (e.g., two) low-temperature heat sources overlapping each other are captured in a thermal image will be described. FIG. 9 is a flowchart illustrating Example 4 of the detailed flow of step S05 shown in FIG. 2.

Calculator 124 detects the minimum value and the next smallest temperature value after the minimum value of the temperature value group corresponding to the human region extracted in step S04 in FIG. 2 (S41). Calculator 124 determines whether the minimum value falls below a second temperature value (e.g., 10° C.) (S42). When calculator 124 determines that the minimum value does not fall below the second temperature value (No in S42), calculator 124 calculates the body surface temperature of the person based on the temperature value group corresponding to the human region (S43). Alternatively, when calculator 124 determines that the minimum value falls below the second temperature value (Yes in S42), calculator 124 determines whether the next smallest temperature value after the minimum value falls below the second temperature value (S44).

When calculator 124 determines that the next smallest temperature value after the minimum value does not fall below the second temperature value (No in S44), calculator 124 identifies (not illustrated) a low-temperature value group of temperature values less than or equal to a sum of the minimum value and a second value (e.g., 20° C.), and calculates the body surface temperature of the person based on the remaining temperature value group obtained by excluding the low-temperature value group from the temperature value group (S46). Alternatively, when calculator 124 determines that the next smallest temperature value after the minimum value falls below the second temperature value (Yes in S44), calculator 124 determines whether a pixel indicating the minimum value and a pixel indicating the next smallest temperature value after the minimum value are positioned a predetermined distance apart from each other in a thermal image (S45). Note that the description of the predetermined distance is omitted since the predetermined distance has been described above.

When calculator 124 determines that the pixel indicating the minimum value and the pixel indicating the next smallest temperature value after the minimum value are not positioned the predetermined distance apart from each other (stated differently, not spaced apart with the predetermined space therebetween) in the thermal image (No in S45), calculator 124 identifies (not illustrated) a low-temperature value group of temperature values less than or equal to a sum of the minimum value and the second value (e.g., 20° C.), and calculates the body surface temperature of the person based on the remaining temperature value group obtained by excluding the low-temperature value group from the temperature value group (S46). Alternatively, when calculator 124 determines that the pixel indicating the minimum value and the pixel indicating the next smallest temperature value after the minimum value are positioned the predetermined distance apart from each other (stated differently, spaced apart with the predetermined space therebetween) in the thermal image (Yes in S45), calculator 124 identifies (not illustrated) low-temperature value group 1 of temperature values less than or equal to a sum of the minimum value and the second value (e.g., 20° C.), further identifies (not illustrated) low-temperature value group 2 of temperature values less than or equal to a sum of the next smallest temperature value after the minimum value and the second value, and calculates the body surface temperature of a person based on the remaining temperature value group obtained by excluding low-temperature value group 1 and low-temperature value group 2 from the temperature value group (S47).

As has been described above, when several (here, two) low-temperature object regions are present within a human region (i.e., when several low-temperature objects overlapping each other are captured in a thermal image), human body surface temperature calculation system 200 can identify a low-temperature value group of each of the several low-temperature object regions to exclude these low-temperature value groups from a temperature value group corresponding to the human region. In this way, even though several heat sources different from a person are present in a thermal image, human body surface temperature calculation system 200 is not easily affected by these heat sources in calculating the body surface temperature of the person. Therefore, human body surface temperature calculation system 200 can accurately calculate the body surface temperature of a person.

Note that Examples 3 and 4 of the calculation processes may be performed in parallel.

For example, calculator 124 detects the maximum value, the next largest temperature value after the maximum value, the minimum value, and the next smallest temperature value after the minimum value within a temperature value group corresponding to a human region. Next, calculator 124 determines whether each of the maximum value and the next largest temperature value after the maximum value exceeds the first temperature value, and determines whether each of the minimum value and the next smallest temperature value after the minimum value falls below the second temperature value.

Based on the determination results, calculator 124 calculates the body surface temperature of a person in accordance with the flow shown in FIG. 8 and FIG. 9.

As has been described above, even though one or more high-temperature object regions and one or more low-temperature object regions are present within a human region, human body surface temperature calculation system 200 can exclude high-temperature value groups and low-temperature value groups from a temperature value group corresponding to the human region. In this way, even though several high-temperature heat sources and low-temperature heat sources are present in a thermal image, human body surface temperature calculation system 200 is not easily affected by heat sources other than a person, such as high-temperature objects and low-temperature objects, in calculating the body surface temperature of the person. Therefore, human body surface temperature calculation system 200 can accurately calculate the body surface temperature of a person.

### Advantageous Effects, Etc.

As has been described, human body surface temperature calculation system 200 includes: infrared sensor 10; obtainer 121 that obtains temperature distribution data indicating a temperature distribution in a target space, which is obtained by infrared sensor 10; thermal image generator 122 that generates a thermal image of the target space based on the temperature distribution data obtained by obtainer 121; extractor 123 that extracts, using machine learning model 132, a human region indicating a person captured in the thermal image; and calculator 124 that extracts a temperature value group corresponding to the human region from the temperature distribution data and calculates a body surface temperature of the person based on the temperature value group extracted.

The above-described human body surface temperature calculation system 200 can accurately calculate the body surface temperature of a person present in a target space.

In addition, for example, calculator 124: (i) when the maximum value of the temperature value group exceeds a first temperature value (e.g., 50° C.), identifies a high-temperature value group of temperature values greater than or equal to a difference between the maximum value and a first value (e.g., 10° C.), where the high-temperature value group corresponds to a high-temperature object region indicating a high-temperature object different from the person; and (ii) calculates the body surface temperature of the person based on a remaining temperature value group obtained by excluding the high-temperature value group from the temperature value group.

Even though a person and a high-temperature heat source (the so-called high-temperature object) other than the person overlapping each other are captured in a thermal image, the above-described human body surface temperature calculation system 200 can exclude a high-temperature value group from the temperature value group. Accordingly, human body surface temperature calculation system 200 is not easily affected by a heat source other than a person, such as a high-temperature object present within a human region, in calculating the body surface temperature of the person. Therefore, human body surface temperature calculation system 200 can very accurately calculate the body surface temperature of a person.

Moreover, for example, when (i) the maximum value and the next largest temperature value after the maximum value exceed the first temperature value and (ii) a pixel indicating the maximum value and a pixel indicating the next largest temperature value after the maximum value are positioned a predetermined distance apart from each other in the thermal image, calculator 124 further incorporates, into the high-temperature value group, a temperature value group of temperature values greater than or equal to a difference between the next largest temperature value after the maximum value and the first value.

Even though a person and several (e.g., two) high-temperature heat sources overlapping each other are captured in a thermal image, the above-described human body surface temperature calculation system 200 can exclude high-temperature value groups from the temperature value group. Accordingly, human body surface temperature calculation system 200 is not easily affected by these heat sources in calculating the body surface temperature of the person. Therefore, human body surface temperature calculation system 200 can accurately calculate the body surface temperature of a person.

In addition, for example, calculator 124: (i) when the minimum value of the temperature value group falls below a second temperature value (e.g., 10° C.), identifies a low-temperature value group of temperature values less than or equal to a sum of the minimum value and a second value (e.g., 20° C.), where the low-temperature value group corresponding to a low-temperature object region indicating a low-temperature object different from the person; and (ii) calculates the body surface temperature of the person based on a remaining temperature value group obtained by excluding the low-temperature value group from the temperature value group.

Even though a person and a low-temperature heat source (the so-called low-temperature object) other than the person overlapping each other are captured in a thermal image, the above-described human body surface temperature calculation system 200 can exclude a low-temperature value group from the temperature value group. Accordingly, human body surface temperature calculation system 200 is not easily affected by a heat source other than a person, such as a low-temperature object present within a human region, in calculating the body surface temperature of the person. Therefore, human body surface temperature calculation system 200 can very accurately calculate the body surface temperature of a person.

Moreover, for example, when (i) the minimum value and the next smallest temperature value after the minimum value fall below the second temperature value and (ii) a pixel indicating the minimum value and a pixel indicating the next smallest temperature value after the minimum value are positioned a predetermined distance apart from each other in the thermal image, calculator 124 further incorporates, into the low-temperature value group, a temperature value group of temperature values less than or equal to a sum of the next smallest temperature value after the minimum value and the second value (e.g., 20° C.).

Even though a person and several (e.g., two) low-temperature heat sources overlapping each other are captured in a thermal image, the above-described human body surface temperature calculation system 200 can exclude low-temperature value groups from the temperature value group. Accordingly, human body surface temperature calculation system 200 is not easily affected by these heat sources in calculating the body surface temperature of the person. Therefore, human body surface temperature calculation system 200 can accurately calculate the body surface temperature of a person.

In addition, a human body surface temperature calculation method to be implemented by a computer, such as human body surface temperature calculation system 200 includes: obtaining temperature distribution data indicating a temperature distribution in a target space; generating a thermal image of the target space based on the temperature distribution data obtained in the obtaining; extracting, using a machine learning model, a human region indicating a person captured in the thermal image; and extracting a temperature value group corresponding to the human region from the temperature distribution data and calculating a body surface temperature of the person based on the temperature value group extracted.

The above-described human body surface temperature calculation method can accurately calculate the body surface temperature of a person present in a target space.

### Other Embodiments

Hereinbefore, the human body surface temperature calculation system and the human body surface temperature calculation method have been described, but the present invention is not limited to the above-described embodiment.

For example, although the above-described embodiment has presented, as one example in which several high-temperature object regions are present within a human region, the example in which two high-temperature object regions are present in the human region, the number of several high-temperature object regions is non-limiting. For example, the number of several high-temperature object regions present within a human region may be three or more. In addition, although the above-described embodiment has presented, as one example in which several low-temperature object regions are present within a human region, the example in which two low-temperature object regions are present in the human region, the number of several low-temperature object regions is non-limiting. For example, the number of several low-temperature object regions present within a human region may be three or more. It should also be noted that when one or more high-temperature object regions and one or more low-temperature object regions are present within a human region, the body surface temperature of a person may be calculated by appropriately combining processes exemplified in the above-described embodiment.

Moreover, in the above-described embodiment, the human body surface temperature calculation system is implemented by several devices, but the human body surface temperature calculation system may be implemented as a single device. For example, the human body surface temperature calculation system may be implemented as a single device equivalent to a server device. When the human body surface temperature calculation system is implemented by several devices, elements to be included in the human body surface temperature calculation system may be assigned to several devices in any way.

Furthermore, in the above-described embodiment, a process performed by a particular processor may be performed by another processor. The order of processes may be changed, and the processes may be performed in parallel.

Moreover, in the above-described embodiment, each element may be implemented by executing a software program suitable for the element. Each element may be implemented as a result of a program execution unit, such as a central processing unit (CPU), processor or the like, loading and executing a software program stored in a storage medium such as a hard disk or a semiconductor memory.

Each element may be implemented by a hardware product. For example, each element may be a circuit (or an integrated circuit). These circuits may constitute a single circuit as a whole or may be individual circuits. These circuits may be general-purpose circuits, or dedicated circuits.

Note that general or specific aspects of the present invention may be implemented by a system, a device, a method, an integrated circuit, a computer program, or a computer-readable recording medium such as a CD-ROM. The general or specific aspects of the present invention may also be implemented by an optional combination of a system, a device, a method, an integrated circuit, a computer program, and a recording medium. For example, the present invention may be implemented as the human body surface temperature calculation method to be executed by a computer such as the human body surface temperature calculation system. The present invention may also be a program for causing a computer to execute the human body surface temperature calculation method, or may be a non-transitory computer-readable recording medium in which such a program is stored.

The present invention also encompasses: embodiments achieved by applying various modifications conceivable to those skilled in the art to each embodiment; and embodiments achieved by optionally combining the structural elements and the functions of each embodiment without departing from the spirit of the present invention.

### Reference Signs List

10 infrared sensor
121 obtainer
122 thermal image generator
123 extractor
124 calculator
5 132 machine learning model
200 human body surface temperature calculation system

1. A human body surface temperature calculation system comprising:
an infrared sensor;
an obtainer that obtains temperature distribution data indicating a temperature distribution in a target space, the temperature distribution data being obtained by the infrared sensor;
a thermal image generator that generates a thermal image of the target space based on the temperature distribution data obtained by the obtainer;
an extractor that extracts, using a machine learning model, a human region indicating a person captured in the thermal image; and
a calculator that extracts a temperature value group corresponding to the human region from the temperature distribution data and calculates a body surface temperature of the person based on the temperature value group extracted.

2. The human body surface temperature calculation system according to claim 1, wherein
the calculator:
when a maximum value of the temperature value group exceeds a first temperature value, identifies a high-temperature value group of temperature values greater than or equal to a difference between the maximum value and a first value, the high-temperature value group corresponding to a high-temperature object region indicating a high-temperature object different from the person; and
calculates the body surface temperature of the person based on a remaining temperature value group obtained by excluding the high-temperature value group from the temperature value group.

3. The human body surface temperature calculation system according to claim 2, wherein
when (i) the maximum value and a next largest temperature value after the maximum value exceed the first temperature value and (ii) a pixel indicating the maximum value and a pixel indicating the next largest temperature value after the maximum value are positioned a predetermined distance apart from each other in the thermal image, the calculator further incorporates, into the high-temperature value group, a temperature value group of temperature values greater than or equal to a difference between the next largest temperature value after the maximum value and the first value.

4. The human body surface temperature calculation system according to claim 1, wherein
the calculator:
when a minimum value of the temperature value group falls below a second temperature value, identifies a low-temperature value group of temperature values less than or equal to a sum of the minimum value and a second value, the low-temperature value group corresponding to a low-temperature object region indicating a low-temperature object different from the person; and
calculates the body surface temperature of the person based on a remaining temperature value group obtained by excluding the low-temperature value group from the temperature value group.

5. The human body surface temperature calculation system according to claim 4, wherein
when (i) the minimum value and a next smallest temperature value after the minimum value fall below the second temperature value and (ii) a pixel indicating the minimum value and a pixel indicating the next smallest temperature value after the minimum value are positioned a predetermined distance apart from each other in the thermal image, the calculator further incorporates, into the low-temperature value group, a temperature value group of temperature values less than or equal to a sum of the next smallest temperature value after the minimum value and the second value.

6. A human body surface temperature calculation method comprising:
obtaining temperature distribution data indicating a temperature distribution in a target space;
generating a thermal image of the target space based on the temperature distribution data obtained in the obtaining;
extracting, using a machine learning model, a human region indicating a person captured in the thermal image; and
extracting a temperature value group corresponding to the human region from the temperature distribution data and calculating a body surface temperature of the person based on the temperature value group extracted.

7. A non-transitory computer-readable recording medium for use in a computer, the recording medium having recorded thereon a computer program for causing the computer to execute the human body surface temperature calculation method according to claim 6.
